(19) United States
(12) Patent Application Publication
(10) Pub. No.: US 2003/0120256 A1
(43) Pub. Date: Jun. 26, 2003

(54) Methods and apparatus for sclerosing the wall of a varicose vein

(75) Inventors: Banning G. Lary, Miami, FL (US); Leonard Pinchuk, Miami, FL (US); Kevin W. Smith, Coral Gables, FL (US); Charles S. Termin, Coral Gables, FL (US)

Correspondence Address: David P. Gordon, Esq., 65 Woods End Road, Stamford, CT, 06905, US

(73) Assignee: SYNTHEON, LLC

(21) Appl. No.: 10/358,523

(22) Filed: Feb. 5, 2003

Related U.S. Application Data
(63) Continuation-in-part of application No. 09/898,867, filed on Jul. 3, 2001.

(51) Int. Cl. A61M 31/00

(52) U.S. Cl. 604/509; 604/102.02

(57) ABSTRACT

Apparatus for sclerosing the wall of a varicose vein includes an inner tube having an expandable balloon at its distal end, an intermediate tube slidably disposed over the inner tube and having a fluid outlet at its distal end and a plunger at its proximal end, and an outer tube through which the plunger is movable. Methods of using the apparatus include deploying the inner and intermediate tubes in the vessel to be sclerosed, inflating the balloon, filling the outer tube with sclerosing agent and moving the plunger from the distal end of the outer tube toward the proximal end.

This application is a continuation-in-part of application Ser. No. 09/898,867 filed Jul. 3, 2001, the complete disclosure of which is hereby incorporated by reference herein.

### BACKGROUND OF THE INVENTION

1. Field of the Invention

The invention relates to the treatment and correction of venous insufficiency or varicose veins. More particularly the invention relates to a minimally invasive procedure using a catheter-based system to sclerose the wall of the vein.

2. State of the Art

The human venous system of the lower limbs consists essentially of the superficial venous system and the deep venous system with perforating veins connecting the two systems. The superficial system includes the long or great saphenous vein and the short saphenous vein. The deep venous system includes the anterior and posterior tibial veins which unite to form the popliteal vein, which in turn becomes the femoral vein when joined by the short saphenous vein.

The venous systems contain numerous one-way valves for directing blood flow back to the heart. Venous valves are usually bicuspid valves, with each cusp forming a sack or reservoir for blood which, under pressure, forces the free surfaces of the cusps together to prevent retrograde flow of the blood and allow antegrade flow to the heart. An incompetent valve is a valve which is unable to close because the cusps do not form a proper seal and retrograde flow of blood cannot be stopped.

Incompetence in the venous system can result from vein dilation. Separation of the cusps of the venous valve at the commissure may occur as a result. Two venous diseases which often involve vein dilation are varicose veins and chronic venous insufficiency.

The varicose vein condition includes dilatation and tortuosity of the superficial veins of the lower limb, resulting in unsightly discoloration, pain and ulceration. Varicose veins often involve incompetence of one or more venous valves, which allow reflux of blood from the deep venous system to the superficial venous system or reflux within the superficial system.

Varicose veins are compatible with long life and rarely cause fatal complications, but the condition significantly decreases the quality of life. Patients complain primarily of leg fatigue, dull, aching pains, ankle swelling, and ulcerations. Occasionally, thrombosis occurs in dilated subcutaneous channels, resulting in local pain, induration, edema, inflammation, and disability. In addition to those problems, the high visibility of the unattractive rope-like swellings and reddish skin blotches causes considerable distress for both men and women. Lastly, varicose eczema, which is a local reddened swollen and itching skin condition can occur and can spread to distant parts of the body (called an “Id reaction”).

Phlebosclerosis, the destruction of venous channels by the injection of sclerosing agents, has been used to treat varicose veins since 1853, when Cassaignae and Ebout used ferric chloride. Sodium salicylate, quinine, urea, and sodium chloride have also been used, but the agent more recently favored is sodium tetradecyl sulfate. In order for phlebosclerosis to be effective, it is necessary to evenly dispense the sclerosing agent throughout the wall of the vein without using toxic levels of the sclerosing agent. This is not particularly difficult for the smaller veins. However, it is quite difficult or nearly impossible in larger veins. When a larger vein is injected with a sclerosing agent, the sclerosing agent is quickly diluted by the substantially larger volume of blood which is not present in smaller veins. The result is that the vein is sclerosed (injured) only in the vicinity of the injection. If the procedure is continued, and the injections are far apart, the vein often assumes a configuration resembling sausage links. The problem cannot be cured by injecting a more potent solution of sclerosing agent, because the sclerosing agent may become toxic at such a concentration.

U.S. Pat. No. 5,676,962 discloses an injectable microfoam containing a sclerosing agent. The microfoam is injected into a vein where it expands and, theoretically, achieves the same results as a larger quantity of sclerosing agent without the toxicity. Such a foam is presently manufactured under the trademark Varisolve® by Provensis, Ltd., London, England. Recent clinical trials of the foam indicate a success rate of 81%.

Until recently, the preferred procedure for treating the great saphenous vein was surgical stripping. This highly invasive procedure involves making a 2.5 cm incision in the groin to expose the saphenofemoral junction, where the great saphenous vein and its branches are doubly ligated en masse with a heavy ligature. The distal portion of the vein is exposed through a 1 cm incision anterior to the medial malleolus, and a flat metal or plastic stripper is introduced to exit in the proximal saphenous vein. The leg is held vertically for 30 seconds to empty the venous tree before stripping the vein from the ankle to the groin. If the small saphenous vein is also incompetent, it is stripped at the same time from an incision posterior to the lateral malleolus to the popliteal space. After stripping the veins, the leg is held in the vertical position for three to four minutes to permit broken vessel ends to retract, constrict, and clot.

After the stripping procedure, collateral veins are removed by the avulsion-extraction technique. By working through small (5 to 8 mm) transverse incisions, segments of vein 10 to 20 cm long can be removed by dissecting subcutaneously along the vein with a hemostat, and then grasping, avulsing, and removing the vein. With practice, long segments of vein in all quadrants can be removed through these small incisions. No attempt is made to ligate the branches or ends of the veins, since stripping has shown it to be unnecessary. Bleeding is controlled by elevation and pressure for two to four minutes. As many as 40 incisions are made in severe cases, but their small size and transverse direction permit closure with a single suture.

Before closure of the incisions, a rolled towel is rolled repeatedly from the knee to the ankle and from the knee to the groin to express any clots that may have accumulated. The groin incision is approximated with three 5-0 nylon mattress sutures and all other incisions are closed with a single suture.

As can be readily appreciated, the stripping and avulsion-extraction procedures are relatively invasive and require significant anaesthesia. It can therefore be appreciated that it would be desirable to provide an alternative, less invasive procedure which would accomplish the same results as stripping and avulsion-extraction.

Recently, a number of patents have issued disclosing the treatment of varicose veins with RF energy. Illustrative of these recent patents are: U.S. Pat. No. 6,200,312 entitled “Expandable Vein Ligator Catheter Having Multiple Electrode Leads”; U.S. Pat. No. 6,179,832 entitled “Expandable Catheter Having Two Sets of Electrodes”; U.S. Pat. No. 6,165,172 entitled “Expandable Vein Ligator Catheter and Method of Use”; U.S. Pat. No. 6,152,899 entitled “Expandable Catheter Having Improved Electrode Design, and Method for Applying Energy”; U.S. Pat. No. 6,071,277 entitled “Method and Apparatus for Reducing the Size of a Hollow Anatomical Structure”; U.S. Pat. No. 6,036,687 entitled “Method and Apparatus for Treating Venous Insufficiency”; U.S. Pat. No. 6,033,398 entitled “Method and Apparatus for Treating Venous Insufficiency Using Directionally Applied Energy”; U.S. Pat. No. 6,014,589 entitled “Catheter Having Expandable Electrodes and Adjustable Stent”; U.S. Pat. No. 5,810,847 entitled “Method and Apparatus for Minimally Invasive Treatment of Chronic Venous Insufficiency”; U.S. Pat. No. 5,730,136 entitled “Venous Pump Efficiency Test System And Method”; and U.S. Pat. No. 5,609,598 entitled “Method and Apparatus for Minimally Invasive Treatment of Chronic Venous Insufficiency”. These patents generally disclose a catheter having an electrode tip which is switchably coupled to a source of RF energy. The catheter is positioned within the vein to be treated, and the electrodes on the catheter are moved toward one side of the vein. RF energy is applied to cause localized heating and corresponding shrinkage of the adjacent venous tissue. After treating one section of the vein, the catheter can be repositioned to place the electrodes to treat different sections of the vein.

Although this procedure has gained acceptance and is less invasive than the stripping and avulsion-extraction procedures, there are several disadvantages to it. In particular, RF treatment is actually quite slow and painful and the patient must be sufficiently anaesthetized along the entire length of the veins to be treated. In addition, repositioning the catheter is time consuming thus requiring anaesthesia for a prolonged period. Moreover, the RF treatment is incomplete, as only a portion of the vein wall is actually treated, i.e. the portion contacting the electrode. The partially treated vein may eventually re-cannularize. Furthermore, tributary veins remain unaffected and must be treated separately. In addition, for even and consistent cauterization, RF treatment requires that the practitioner be keenly aware of the procedure time. If RF energy is applied for too long, it can cause undesired burns. If RF energy is not applied long enough, the treatment is ineffective.

In addition to RF treatment, laser treatment has been used with some success. Laser treatment shares many of the disadvantages of RF treatment. In particular, as with the RF devices, the practitioner must be very careful as to the intensity and duration of the treatment to assure that the treatment is effective but without causing undesired burns.

Our previously incorporated parent application discloses an apparatus for delivering an intravascular drug such as a sclerosing agent (or a microfoam sclerosing agent) to a varicose vein which includes a catheter having three concentric tubes. The innermost tube has a guide wire lumen and an inflation lumen. The distal end of the innermost tube has an integral inflatable occlusion balloon in fluid communication with the inflation lumen. The intermediate tube has a lumen through which the innermost tube extends. The distal end of the intermediate tube has a self-expanding balloon with a plurality of fluid pores in fluid communication with the intermediate tube lumen. The outer tube has a lumen through which the intermediate tube extends. Sclerosing agent is dispensed through the intermediate tube to pores located at the distal end of the intermediate tube or in the self-expanding balloon. Veins are sclerosed as the self-expanding balloon is pulled through and ultimately out of the vein.

Subsequent development of the apparatus of the parent application resulted in several improvements.

### SUMMARY OF THE INVENTION

It is therefore an object of the invention to provide methods and apparatus for the minimally invasive treatment of varicose veins.

It is also an object of the invention to provide methods and apparatus for the minimally invasive treatment of varicose veins wherein only minimal anaesthesia is required.

It is another object of the invention to provide methods and apparatus for the minimally invasive treatment of varicose veins wherein tributary veins are treated simultaneously with the vein to which they connect.

It is an additional object of the invention to provide methods and apparatus for the minimally invasive treatment of varicose veins and connecting tributaries wherein the entire wall of the vein is evenly sclerosed.

Another object of the invention is to provide methods and apparatus for the minimally invasive treatment of varicose veins which do not utilize high concentration sclerosing agents.

Yet another object of the invention is to provide methods and apparatus for the minimally invasive treatment of varicose veins which do not require that the practitioner carefully monitor the duration, rate, or progression of treatment.

Still another object of the invention is to improve upon the methods and apparatus of the previously incorporated parent application.

In accord with these objects which will be discussed in detail below, an apparatus according to the present invention includes a catheter device having three concentric tubes: an inner tube, an outer tube, and an intermediate tube. Each tube has a proximal end and a distal end with a lumen extending therethrough. As used herein, the term proximal means closest to the practitioner and the term distal means farthest from the practitioner when the apparatus is in use. An inflatable balloon is located at or near the distal end of inner tube and a fluid valve is coupled to the proximal end of the inner tube. The balloon is inflated by injecting fluid through the valve and is held in an inflated condition by closing the valve. A fluid outlet is located at or near the distal end of the intermediate tube and a “plunger” is coupled to the proximal end of the intermediate tube. The plunger is movable within the outer tube defining a fluid reservoir of varying size between the proximal end of the outer tube and the plunger. The plunger permits fluid communication between the fluid reservoir and the lumen of the intermediate tube. The proximal end of the outer tube is provided with a trifurcated fitting including a Touhy-Borst type connector. The proximal end of the inner tube extends through the Touhy-Borst connector which provides a fluid seal between the inner tube and the outer tube and which locks the inner tube in position relative to the outer tube. A pullwire is coupled to the plunger and extends through a central port of the trifurcated fitting which maintains a fluid seal between the pullwire and the outer tube. The third port of the trifurcated fitting is provided with a female Luer with a check valve which permits one-way fluid access into the fluid reservoir. According to the presently preferred embodiment, the distal end of the inner tube is provided with a radiopaque tip and a safety wire extends within the inner tube providing the inner tube with stiffness and maneuverability for precise placement of the inflatable balloon. The wire is bonded to or captures the entire device, thereby helping to keep it together. Further according to the presently preferred embodiment, the outer tube is transparent and provided with a plurality of movable exterior markers which are useful in performing the methods of the invention.

According to alternate embodiments of the apparatus, other types of tracking devices may be used at the tip of the inner tube rather than the radiopaque tip. Examples of such devices include an LED or an illuminated fiber optic which is visible through the skin, or a magnet which can be detected with an electromagnetic sensor.

Methods of the invention include examining the patient and marking the patient's leg to indicate the entry site, the occlusion site and important sites (e.g. tributaries) along the blood vessel. The distal end of the outer tube is placed adjacent to the entry site and the inner tube and intermediate tube are extended outside the patient along the leg to the occlusion site. The intermediate tube is then drawn back from the occlusion site to the first important site marking proximal of the occlusion site. One of the movable exterior markers on the outer tube is then moved to the position occupied by the plunger. The intermediate tube is then moved to the next proximal important site marking on the leg and another marker on the outer tuber is moved to the corresponding position of the plunger. These steps are repeated until all of the important site markings have been recorded with the movable markers on the outer tube. The catheter is then reset so that the distal ends of the inner tube and intermediate tube are adjacent to each other. A 10 cc-20 cc syringe is loaded with sclerosing agent and is attached to the female luer. While holding the catheter in an upward direction, 10 cc of sclerosing agent is injected into the fluid reservoir and the intermediate tube until a few drops exit the fluid outlet of the intermediate tube and the tubes are purged of air bubbles. If necessary, the syringe is reloaded with additional sclerosing agent.

The inner and intermediate tubes are then inserted through a hemostasis valve or cut-down into the blood vessel and maneuvered through the vessel until the distal end of the outer tube abuts the vessel or hemostasis valve. The balloon is then inflated using a 3 cc-5 cc syringe coupled to the proximal end of the inner tube. Infusion of sclerosing agent is commenced by pulling the pullwire so that the plunger is moved proximally forcing fluid out of the fluid reservoir through the intermediate tube and out of the fluid outlets at the distal end of the intermediate tube. When the plunger reaches one of the markers on the outer tube, additional sclerosing agent may be injected using the 10 cc-20 cc syringe. The plunger is then moved to the next marker and additional sclerosing agent is injected. After all of the markers have been passed by the plunger, the balloon is deflated and the catheter device is removed from the patient.

Additional objects and advantages of the invention will become apparent to those skilled in the art upon reference to the detailed description taken in conjunction with the provided figures.

### BRIEF DESCRIPTION OF THE DRAWINGS

FIG. 1 is a schematic side elevational view of a catheter device according to the invention with the inner and intermediate tubes withdrawn;

FIG. 2 is a schematic side elevational view of a catheter device according to the invention with the inner and intermediate tubes extended;

FIG. 3 is a schematic side elevational view of a catheter device according to the invention in use; and

FIGS. 4a-4e are schematic illustrations of the distal ends of the inner tube and intermediate tube during use.

### DETAILED DESCRIPTION OF THE PREFERRED EMBODIMENTS

1. The Apparatus According to the Invention

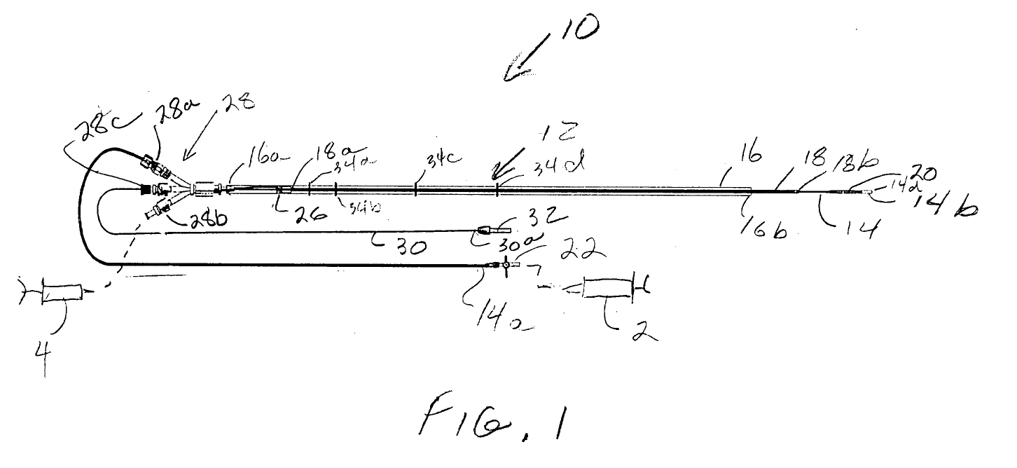

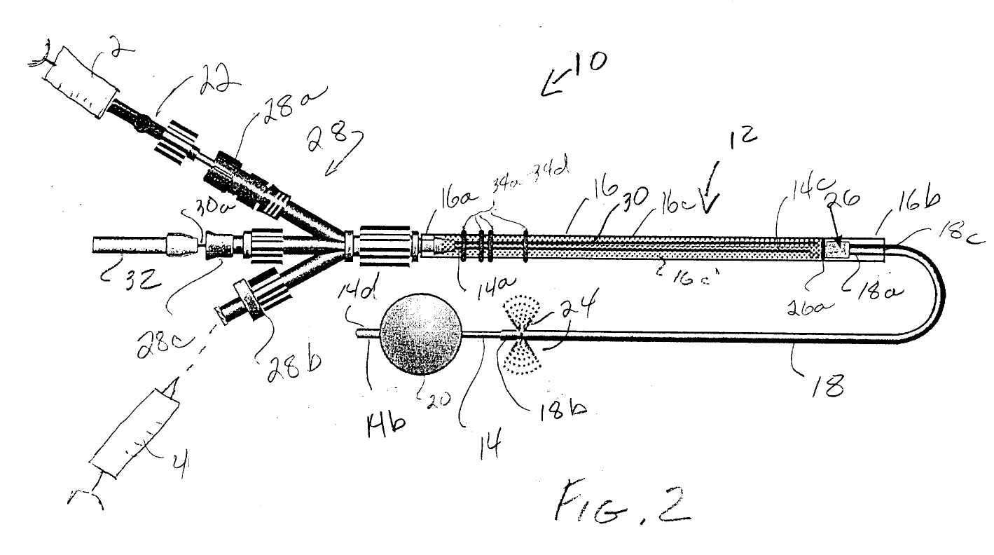

Referring now to FIGS. 1 and 2, an apparatus 10 according to the present invention includes a catheter device 12 having three concentric tubes: an inner tube 14, an outer tube 16, and an intermediate tube 18. Each tube 14, 16, 18 has a proximal end 14a, 16a, 18a and a distal end 14b, 16b, 18b with a lumen 14c, 16c, 18c extending therethrough. As used herein, the term proximal means closest to the practitioner and the term distal means farthest from the practitioner when the apparatus is in use.

An inflatable balloon 20 is located at or near the distal end 14b of inner tube 14 and a fluid valve 22 is coupled to the proximal end 14a of the inner tube 14. The balloon 20 is inflated by injecting fluid (e.g. saline) through the valve 22 and is held in an inflated condition by closing the valve 22.

As seen best in FIG. 2, one or more fluid outlet(s) 24 are located at or near the distal end 18b of the intermediate tube 18 and a “plunger” 26 is coupled to the proximal end 18a of the intermediate tube 18. According to the presently preferred embodiment, the fluid outlets 24 include a plurality of radial outlets and a fluid seal (not shown) closes the annular space between the tube 14 and the tube 18 at a location distal of the outlets 24. The fluid seal (not shown) is heat formed and makes a sliding (dynamic) seal. The plunger 26 is movable within the outer tube 16 defining a fluid reservoir 16c′ of varying size between the proximal end 16a of the outer tube 16 and the plunger 26. For example, FIGS. 1 and 2 illustrate two extreme locations of the plunger 26, FIG. 1 showing a small reservoir and FIG. 2 showing a large reservoir. The plunger 26 permits fluid communication between the fluid reservoir 16c′ and the lumen 18c of the intermediate tube 18. According to the presently preferred embodiment, the plunger 26 is provided with an indication 26a as seen best in FIG. 2. According to the presently preferred embodiment, the indication 26a is a sealing O-ring contrasting in color to that of the plunger 26.

The proximal end 16a of the outer tube 16 is provided with a trifurcated fitting 28 including a Touhy-Borst type connector 28a, a female Luer 28b with check valve (not shown) and a Luer 28c housing a seal connector (not shown).

The proximal end 14a of the inner tube 14 extends through the Touhy-Borst connector 28a which provides a fluid seal between the inner tube 14 and the outer tube 16 and which selectively locks the inner tube 14 in position relative to the outer tube 16.

The female Luer 28b with check valve permits one-way fluid access into the fluid reservoir 16c′ of the outer tube 16.

A pullwire 30 is coupled to the plunger 26 and extends through the luer 28c of the trifurcated fitting 28 which maintains a fluid seal between the pullwire 30 and the outer tube 16. The proximal end 30a of the pullwire 30 is provided with a handle 32. According to the presently preferred embodiment, the handle is a striking color (e.g. orange) so that it can be quickly located.

According to the presently preferred embodiment, the distal end 14b of the inner tube 14 is provided with a radiopaque tip 14d and a safety wire (not shown in FIGS. 1 or 2) extends within the inner tube 14 providing the inner tube with stiffness and maneuverability for precise placement of the inflatable balloon.

Further according to the presently preferred embodiment, the outer tube 16 is transparent and provided with a plurality of movable exterior markers 34a-34d which are used in conjunction with the indication 26a on the plunger 26 in performing the methods of the invention described in more detail below. The presently preferred markers are elastic O-rings.

According to alternate embodiments of the apparatus, other types of tracking devices may be used at the distal end of the inner tube rather than the radiopaque tip. Examples of such devices include an LED or an illuminated fiber optic which is visible through the skin, or a magnet which can be detected with an electromagnetic sensor.

The apparatus 10 is intended for use with and thus also preferably includes two syringes, a 3-5 cc syringe 2 for inflating the balloon and a 10-20 cc syringe 4 for injecting sclerosing agent.

2. Recommended Procedures According to the Invention

Although it is not necessary to perform the procedure in an operating room, it is considered prudent for the initial examination to be performed in an out-patient suite in a hospital or in an operating room in the event that any unforeseen events occur that may require surgical intervention.

The patient should first be examined under ultrasound, palpation, fluoroscopy or other means for venous valve insufficiency and varicose veins. If the physician determines that the patient is a candidate for closure of the saphenous vein as a means of eliminating the varicosities, the patient will be admitted for the procedure.

Preferably, a photograph of the patient's leg is taken both before and after the procedure so that the results of the procedure can be readily ascertained.

The patient is preferably sedated with a mild sedative such as Percocet, or the like, one hour prior to the procedure. An IV line may be inserted in the patient's arm and vital signs monitored throughout the procedure.

While the patient is standing, the saphenofemoral junction is located using Doppler or other ultrasonic techniques and the skin marked over this junction with a washable marker. Similarly, the saphenous vein and its major tributary junctions is traced using ultrasound and its path marked on the surface of the skin with a marker.

If varicosities are present above the knee only, then the length of the saphenous vein from the knee to the groin will be treated either through a cut down to the saphenous vein or by a percutaneous stick into the saphenous vein (or both) using a catheter sheath introducer. If the disease is prevalent below the knee, then a similar incision or percutaneous stick will be made in the saphenous vein at the level of the ankle and the vein sclerosed from the ankle to the knee. If the disease is prevalent in both the upper and lower leg, then an incision or percutaneous stick will be made in the saphenous vein at the level of the ankle and the vein sclerosed from the ankle to the groin and the entire vein sclerosed.

The patient lies down with his/her leg elevated 30 to 45 degrees to allow blood to drain from the leg. The patient's leg is scrubbed with a standard surgical preparation medium, such as betadine and the site prepared for an aseptic procedure. Lidocaine or other local anesthetic is injected into the area around the vein with a small needle.

Prior to use, the apparatus 10 should be examined to determine that it is functioning properly. This should include sliding the plunger in and out through the outer tube and dilating the balloon with 3 cc of sterile saline.

The following procedure assumes that the patient's skin has been previously marked with the entry site, the occlusion site and important sites (e.g. tributaries) along the vessel. It also assumes that the catheter device can be laid down on the patient's leg while maintaining sterility.

With the inner tube 14 and the intermediate tube 18 drawn into the outer tube 16 as shown in FIG. 1, the distal end 16b of the outer tube 16 is located at the entry site (just proximal to the hemostasis valve of the CSI). While the outer tube 16 is maintained in position, the inner tube 14 and the intermediate tube 18 are pulled out of the outer tube 16, by grasping and pulling the intermediate tube, until the balloon 20 is over the mark on the skin representing the occlusion site.

The inner tube 14 is locked in position by tightening the Touhy Borst valve 28a. Locking the Touhy Borst valve assures that when the apparatus is inserted into the leg, the balloon will inflate at the desired occlusion site. It also assures that the balloon will not migrate backwards when the sclerosing agent is dispensed.

Starting with the distal end 18b of the intermediate tube 18 abutting the balloon 20, the pullwire 30 is pulled such that the intermediate tube moves backwards until the fluid outlet 24 is located at the next marking on the patient's leg (e.g. a tributary site). With the apparatus in this position, the closest marker (o-ring) 34d is moved over the tube 16 until it is aligned with the indicia 26a on the plunger 26. The pullwire 30 is pulled again and this step is repeated for each of the marks on the patient's leg, using the o-rings 34c, 34b, 34a to mark the corresponding location of the plunger 26. It will be appreciated that the number of markers shown in the Figures is arbitrary and more or fewer markers may be provided.

After all of the desired markers 34a-34d have been placed along the tube 16, the intermediate tube 18 is pulled distally until its distal end 18b abuts the balloon 20 as shown in FIG. 2.

As mentioned above, two syringes are used to operate the apparatus, a 3-5 cc syringe 2 to expand the balloon and a 10-20 cc syringe 4 to dispense the sclerosing agent. The smaller syringe is filled with sterile saline and attached to the fluid valve 22 (a Luer with a stop cock). The larger syringe is filled with sclerosing agent and attached to the female Luer 28b. While holding the intermediate tube 18 in an upward direction, 10 cc of the sclerosing agent is injected through the check valve 28b into the reservoir 16c′ of the tube 16, through the plunger 26, and up through the tube 18 such that a few drops of fluid emerge from the fluid outlets 24 on the distal end of the tube 18. The physician should ensure that the tubes 16, 18 are purged of air bubbles. If necessary, the larger syringe is reloaded with additional sclerosing agent before proceeding.

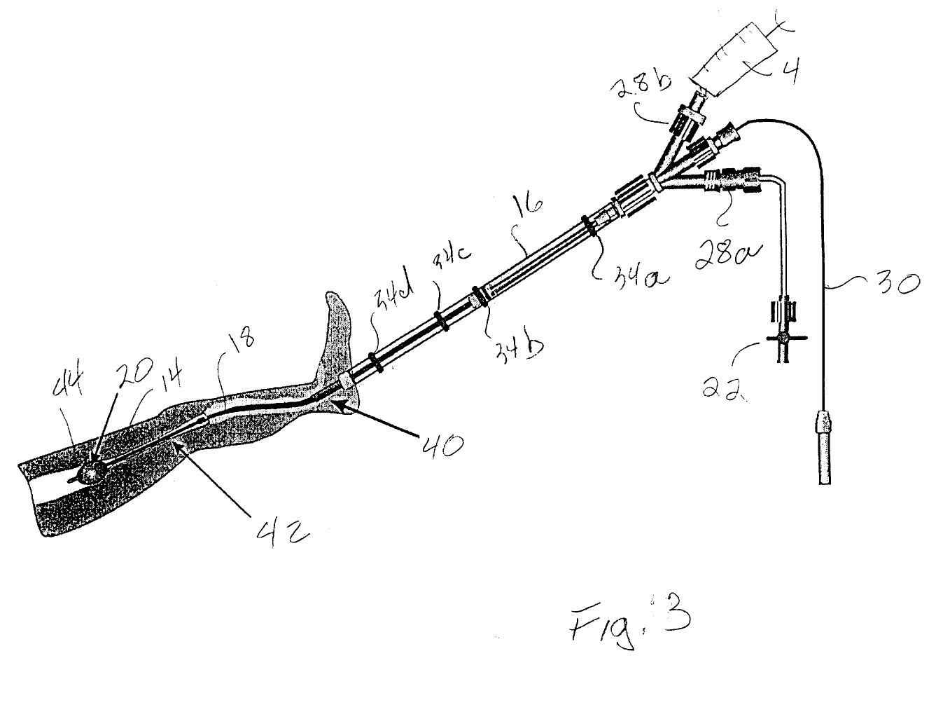

The inner tube 14 and the intermediate tube 18 are then inserted into a percutaneous stick 40 in the saphenous vein 42 as shown in FIG. 3. The tubes 14, 18 are maneuvered to the occlusion location 44 preferably with the aid of the tip indicator 14d of the tube 14. As mentioned above, the tip indicator 14d may be radiopaque and thus located with fluoroscopy. Alternatively, the tip 14d may be provided with an LED or an optical fiber which causes it to glow bright enough to be seen through the skin. Still alternatively, the tip 14d may be magnetic and thus located with electromagnetic equipment.

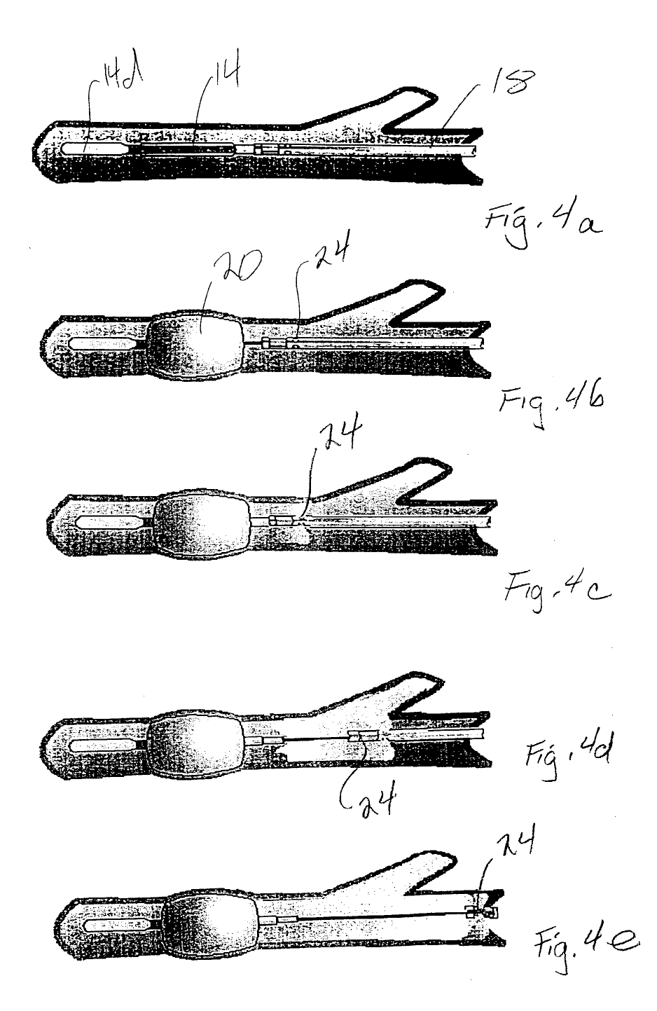

With the apparatus in position as shown in FIG. 4a, the balloon 20 is expanded with the small syringe as shown in FIG. 4b. According to the presently preferred embodiment, preferably no more than 5 cc should be injected into the balloon which will expand to a diameter of approximately 21 mm upon injection of 5 cc. Table 1 illustrates a typical relationship between the injection volume and the balloon diameter.

TABLE 1

| Injection volume ± 0.1 cc | Balloon Diameter ± 1 mm |
|---|---|
| 1 | 12 |
| 2 | 15 |
| 3 | 18 |
| 4 | 19 |
| 5 | 21 |

The balloon is preferably inflated slowly with sterile saline or radiopaque media until it totally occludes the vessel. Ultrasound, fluoroscopy, palpation, tugging, etc. can be used to ensure that the balloon is adequately inflated. Once the balloon is inflated, the stopcock 22 is closed by rotating the stopcock 90°.

The infusion procedure is begun by pulling the pullwire 30 back until the O-ring on the piston lines up with the first O-ring marker previously located on the tube 16. Pulling on the pullwire causes the plunger 26 to be moved toward the proximal end of the tube 16, which in turn forces the sclerosing agent out of the fluid outlets 24 in the distal end of the tube 18 which is also moved away from the balloon 20 as shown in FIG. 4c. This releases a controlled and evenly distributed amount of sclerosing agent which is well suited for sclerosing a vein with no tributaries. When the end of the tube 18 reaches a tributary, as shown in FIG. 4d and as indicated by the placement of the O-rings 34a-34d, it is desirable to release additional sclerosing agent to contract the tributary as well as the vein. This may be accomplished by injecting additional sclerosing agent with the large syringe which remains attached to the injection port 28b. After the additional sclerosing agent is released, movement of the tube 18 is resumed as shown in FIG. 4e.

Injection of this bolus of sclerosing agent may be directed and facilitated with a fork-like device (not shown) that compresses the outside of the leg on either side of the fluid outlets 24. A roller may also be used to force the sclerosing agent up the tributary. This process is repeated for other large tributaries. Preferably no more than 20 cc of 0.5% sclerosing agent should be used in this procedure.

When the tube 18 is fully withdrawn, the balloon 20 is deflated by aspiration and the tube 14 is removed from the vein. The entry site may be sutured before dressing. However, according to the presently preferred embodiment, the size of the introducer is only 6-French which may produce a sufficiently small wound so as not to require suturing. However, the leg is preferably immediately wrapped in a gauze-type dressing (e.g., KERLIX® available from Kendall Co., Walpole, Mass.). A length of foam rubber padding is preferably placed over the gauze and over the saphenous vein that was sclerosed. An elastic bandage (e.g., COACH® or ACE®) is preferably placed over the foam rubber to keep it in place. An additional elastic bandage may be placed over the first elastic bandage to ensure that the vein remains compressed and that blood does not flow back into the treated veins.

The patient should be advised to rest with his/her leg elevated for approximately 30 minutes. The patient can then walk to the car, elevate the leg in the car and then keep the leg elevated in bed overnight. Occasional flexure of the foot, ankle and leg should be encouraged. It is preferred that the patient be re-examined the following day. The dressings should then be replaced and the patient instructed on how to self apply new dressings and bandages. The dressings, foam pads and bandages may be kept in place for five to seven days. After five to seven days, the patient should be re-examined and, if indicated, the dressings and foam removed. The compression bandage should be worn for an additional week.

The patient should be asked to return for follow-up at one month and three months if indicated. The patient may also be asked to return at one year to evaluate the long term effectiveness of the procedure.

The benefits of the methods and apparatus of the invention include:

Sclerosing agents are painless in the vascular system as compared to laser or RF ablation that can be extremely painful.

The occlusion balloon prevents the sclerosing agent from entering the deep venous system via the saphenofemoral or saphenopoliteal junctions.

The catheter is 6-Fr in diameter and is easily maneuvered through the vein.

Only one injection of anesthesia is required at the puncture site, resulting in less pain and toxicity to the patient.

Venous access via a small cut down or by use of a catheter sheath introducer produces a very minimal scar, resulting in a better cosmetic impact.

The recovery time is faster with fewer cosmetic complications as compared to stripping.

Tributaries can be treated as well as the main veins resulting in a better cosmetic impact.

Veins below the knee can be treated.

The total procedural time is greatly reduced.

The apparatus is less expensive than laser and RF apparatus.

The procedure is performed in an outpatient setting.

The apparatus automatically assures that the correct amount of sclerosing agent is evenly distributed without requiring the practitioner to carefully monitor the duration of treatment.

There have been described and illustrated herein several embodiments of methods and apparatus for sclerosing the wall of a varicose vein. While particular embodiments of the invention have been described, it is not intended that the invention be limited thereto, as it is intended that the invention be as broad in scope as the art will allow and that the specification be read likewise. It will therefore be appreciated by those skilled in the art that yet other modifications could be made to the provided invention without deviating from its spirit and scope as so claimed.

1. An apparatus for delivering an intravascular drug, said apparatus comprising:
a) a first tube having a proximal end, a distal end, and a fluid lumen extending from its proximal end to its distal end;
b) an inflatable balloon coupled to said distal end of said first tube and in fluid communication with said fluid lumen;
c) a second tube having a proximal end, a distal end, and a fluid lumen extending from its proximal end to its distal end, said first tube extending through said fluid lumen of said second tube, said second tube having at least one distal fluid outlet, wherein
said second tube is adapted to receive and deliver the intravascular drug to a location proximal of said inflatable balloon.

2. The apparatus according to claim 1, further comprising:
d) a third tube having a proximal end, a distal end, and a fluid lumen extending from its proximal end to its distal end; and
e) a movable plunger disposed in said third tube, said movable plunger coupled to said proximal end of said second tube and permitting fluid communication from said fluid lumen of said third tube to said fluid lumen of said second tube.

3. The apparatus according to claim 2, wherein:
movement of said plunger toward said proximal end of said third tube causes said distal end of said second tube to move proximally relative to said inflatable balloon.

4. The apparatus according to claim 3, wherein:
said proximal end of said third tube is coupled to means for injecting the intravascular drug into said lumen of said third tube.

5. The apparatus according to claim 4, wherein:
said means for injecting includes one-way valve means for preventing the drug from exiting the lumen of the third tube through said means for injecting.

6. The apparatus according to claim 4, wherein:
when said lumen of said third tube and said lumen of said second tube are filled with the intravascular drug, movement of said plunger toward said proximal end of said third tube causes the intravascular drug to exit said at least one distal fluid outlet.

7. The apparatus according to claim 5, wherein:
said means for injecting includes a syringe coupled to said proximal end of said third tube

8. The apparatus according to claim 1, further comprising:
d) means for inflating said balloon coupled to said proximal end of said first tube.

9. The apparatus according to claim 8, wherein:
said means for inflating includes a syringe.

10. The apparatus according to claim 9, wherein:
said means for inflating includes a stop cock.

11. The apparatus according to claim 2, further comprising:
f) a movable marker on said third tube for indicating a location between said proximal end and said distal end of said third tube.

12. The apparatus according to claim 11, wherein:
said movable marker includes a plurality of elastic o-rings.

13. The apparatus according to claim 11, wherein:
said third tube is substantially transparent or translucent such that said plunger is visible.

14. The apparatus according to claim 11, wherein:
said plunger includes indicia which is visible through said third tube.

15. The apparatus according to claim 1, further comprising:
d) location means for locating the inflatable balloon when said inflatable balloon is located inside a blood vessel.

16. The apparatus according to claim 15, wherein:
said location means includes a radiopaque member coupled to said first tube.

17. The apparatus according to claim 15, wherein:
said location means includes a light emitting member.

18. The apparatus according to claim 17, wherein:
said light emitting member includes an LED.

19. The apparatus according to claim 17, wherein:
said light emitting member includes a fiber optic.

20. The apparatus according to claim 15, wherein:
said location means includes a magnetic member.

21. An apparatus for delivering an intravascular drug, said apparatus comprising:
a) a drug delivery tube having a proximal end, a distal end, and a lumen extending from its proximal end to its distal end;
b) a drug reservoir fluidly coupled to the proximal end of the lumen of the drug delivery tube; and
c) dispensing means coupled to said drug reservoir, said dispensing means being adapted to automatically dispense the drug from the reservoir into the lumen of the drug delivery tube as the drug delivery tube is moved through a blood vessel.

22. The apparatus according to claim 21, wherein:
said drug reservoir includes a tube having a proximal end and a distal end,
said proximal end having drug receiving means for receiving the intravascular drug,
said dispensing means including a plunger coupled to said proximal end of said drug delivery tube and movable within said drug reservoir.

23. The apparatus according to claim 22, wherein:
said dispensing means includes a pullwire coupled to said plunger and extending through said proximal end of said drug reservoir tube.

24. The apparatus according to claim 22, wherein:
said drug receiving means includes a one way valve.

25. The apparatus according to claim 22, wherein:
said drug receiving means includes a female Luer.

26. An apparatus for delivering an intravascular drug, said apparatus comprising:
a) a drug delivery tube having a proximal end, a distal end, and a lumen extending from its proximal end to its distal end;
b) dispensing means coupled to said proximal end of said drug delivery tube for automatically dispense the drug from the distal end of said drug delivery tube as the drug delivery tube is moved through a blood vessel.

27. A method for delivering an intravascular drug, comprising:
a) delivering a drug delivery catheter and an expandable balloon into a blood vessel;
b) expanding the balloon; and
c) partially removing the drug delivery catheter from the blood vessel while dispensing of the drug.

28. The method according to claim 27, wherein;
the drug is dispensed evenly regardless of the speed at which the catheter is removed.

29. The method according to claim 27, wherein:
the balloon is delivered coaxially to the drug delivery catheter.

30. The method according to claim 29, wherein:
the balloon is an inflatable balloon attached to an inflation catheter which is coaxial to the drug delivery catheter.
